(19) United States
(12) Patent Application Publication
(10) Pub. No.: US 2024/0108403 A1
(43) Pub. Date: Apr. 4, 2024

(54) ELECTRODE CATHETER AND METHOD OF MANUFACTURING CATHETER SHAFT

(71) Applicant: Japan Lifeline Co., Ltd., Tokyo (JP)

(72) Inventors: Tomoharu KOISO, Tokyo (JP); Hiroki KAMIYAMA, Tokyo (JP); Shingo HAYASHIDA, Tokyo (JP)

(21) Appl. No.: 18/478,390

(22) Filed: Sep. 29, 2023

(30) Foreign Application Priority Data
Sep. 29, 2022 (JP) 2022-156205

(51) Int. Cl. A61B 18/14; A61M 25/00

(52) U.S. Cl. CPC A61B 18/1492; A61M 25/005; A61B 2018/00077

(57) ABSTRACT

An electrode catheter includes a catheter shaft including a shaft main body enclosing a first lumen and a second lumen separated from each other, and a reinforcement layer embedded into the shaft main body, an electrode having a ring shape and mounted to an outer peripheral portion of the shaft main body, and a lead wire electrically conductive to the electrode and inserted into the first lumen. The reinforcement layer has conductivity and, inside the electrode, surrounds the second lumen without surrounding the first lumen.

### CROSS REFERENCE TO RELATED APPLICATIONS

This application claims the benefit of priority to Japanese Patent Application Number 2022-156205 filed on Sep. 29, 2022. The entire contents of the above-identified application are hereby incorporated by reference.

### TECHNICAL FIELD

The present disclosure relates to an electrode catheter.

### BACKGROUND

JP 2017-148472 A discloses an electrode catheter including a catheter shaft provided with a shaft main body enclosing a lumen, electrodes having ring shapes and mounted to an outer peripheral portion of the catheter shaft, and lead wires electrically conductive to the electrodes and inserted into the lumen. In the electrode catheter of JP 2017-148472 A, in order to improve a torque transmissibility of the catheter shaft, a reinforcement layer made of a resin and composed of braids is embedded into the shaft main body.

### SUMMARY

From the viewpoint of increasing a degree of freedom in design, it is desirable to employ a reinforcement layer having conductivity, such as a metal, instead of a reinforcement layer made of a resin which typically does have conductivity. The inventors of the present application discovered a new idea for avoiding unintended conduction between a reinforcement layer having conductivity and a lead wire while securing a torque transmissibility by the reinforcement layer.

An object of the present disclosure is to provide a technique for avoiding unintended conduction between a reinforcement layer having conductivity and a lead wire while securing a torque transmissibility by the reinforcement layer.

An electrode catheter according to a first aspect of the present disclosure includes a catheter shaft including a shaft main body enclosing a first lumen and a second lumen separated from each other, and a reinforcement layer embedded into the shaft main body, an electrode having a ring shape and mounted to an outer peripheral portion of the shaft main body, and a lead wire electrically conductive to the electrode and inserted into the first lumen. The reinforcement layer has conductivity and, inside the electrode, surrounds the second lumen without surrounding the first lumen.

In an electrode catheter according to a second aspect of the present disclosure, in the first aspect described above, the catheter shaft includes a lumen tube forming the second lumen, the shaft main body is formed of at least one resin layer and encloses the lumen tube, and the reinforcement layer is wound around the lumen tube and embedded into the at least one resin layer covering the lumen tube.

Another aspect of the present disclosure is a method of manufacturing the catheter shaft according to the second aspect. The method includes extrusion-molding the at least one resin layer covering the lumen tube and thus embedding the reinforcement layer, in a state where the reinforcement layer is wound around an outer peripheral portion of the lumen tube.

According to the present disclosure, it is possible to avoid unintended conduction between a reinforcement layer having conductivity and a lead wire while securing a torque transmissibility by the reinforcement layer.

### BRIEF DESCRIPTION OF DRAWINGS

FIG. 1 is a side view of an electrode catheter according to an embodiment.

FIG. 2 is a cross-sectional view taken along II-II in FIG. 1.

FIG. 3 is a cross-sectional view taken along III-III in FIG. 1.

FIG. 4 is a cross-sectional view taken along IV-IV in FIG. 3.

FIG. 5A is a first explanatory view related to a method of manufacturing a catheter shaft.

FIG. 5B is a second explanatory view related to the method of manufacturing the catheter shaft.

FIG. 6 is an enlarged view around a narrow portion of FIG. 3.

FIG. 7 is an explanatory view of a cross-sectional shape of an element wire.

FIG. 8 is a cross-section view of the electrode catheter according to a reference embodiment.

### DESCRIPTION OF EMBODIMENTS

Hereinafter, embodiments for carrying out the present disclosure will be described. The same or equivalent constituent elements are denoted by the same reference signs, and redundant descriptions are omitted. In the drawings, for convenience of explanation, constituent elements are omitted, enlarged, or reduced, as appropriate. The drawings are to be viewed in accordance with the orientation of the reference signs.

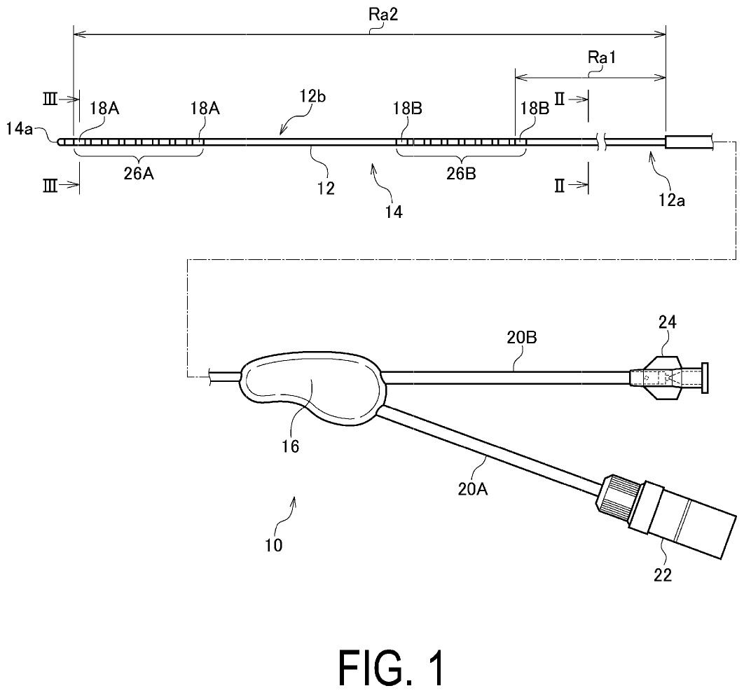

Referring to FIG. 1, an electrode catheter 10 is used for treatment of an organ of a living body. The term “treatment” as used herein refers to the treatment of a disease, an injury, or the like. Here, an example in which a treated portion of this organ is a cardiac cavity portion of a heart will be described. Here, the cardiac cavity portion refers to a location including an atrium such as the right atrium or the left atrium and a ventricle such as the right ventricle or the left ventricle. When the electrode catheter 10 is used for treatment of the cardiac cavity portion, there is a superior vena cava approach in which the electrode catheter 10 is inserted into the coronary sinus from the superior vena cava via the right atrium, and an inferior vena cava approach in which the electrode catheter 10 is inserted into the coronary sinus from the inferior vena cava via the right atrium. The electrode catheter 10 may be used with any of these approaches.

The electrode catheter 10 includes a catheter shaft 14 provided with a shaft main body 12, a handle 16 attached to a proximal end side portion 12a of the shaft main body 12, and a plurality of electrodes 18A, 18B having ring shapes and mounted to an outer peripheral portion of a distal end side portion 12b of the shaft main body 12. Hereinafter, when reference is simply made to an axial direction, a radial direction, and a circumferential direction, the terms refer to the axial direction, the radial direction, and the circumferential direction of the catheter shaft 14.

The catheter shaft 14 has elastically deformable flexibility. A distal end tip 14a for protecting the catheter shaft 14 is mounted onto a distal end portion of the catheter shaft 14. An outer diameter of the catheter shaft 14 is, for example, 7 Fr (2.3 mm) or less, preferably 6 Fr (2.0 mm) or less, when used in a heart of a living body.

The handle 16 is gripped by a practitioner such as a doctor. When torque is applied to the proximal end side portion 12a of the shaft main body 12 by operation of the handle 16, the torque is transmitted to the distal end side portion 12b of the shaft main body 12. A connector 22 for electrical connection to an external power supply device (not illustrated) is attached, via a first protective tube 20A, to the handle 16 of the present embodiment. A port member 24 communicating with an interior of a second lumen 34 (described below) of the shaft main body 12 is attached, via a second protective tube 20B, to the handle 16 of the present embodiment.

The electrodes 18A, 18B are constituted by a metal having favorable electrical conductivity, such as platinum, gold, silver, aluminum, copper, and stainless steel, for example. The plurality of electrodes 18A, 18B are used to output electricity supplied for treatment of a treated portion of a living body. The plurality of electrodes 18A, 18B are not used only for measurement of cardiac potential or the like. In other words, the plurality of electrodes 18A, 18B are not used only for the incorporation of weak electric signals emitted from the living body. The electricity supplied for this treatment is generated by the external power supply device and then output, via the plurality of electrodes 18A, 18B, from the external power supply device to the treated portion of the living body. Here, “electricity supplied for treatment” refers to electricity supplied for treatment such as, for example, defibrillation or ablation (pulsed field ablation (PFA) or high-frequency ablation). The treatment mode described here is merely an example, and the present disclosure may be applied to various treatments that can be realized by the output of electricity.

The plurality of electrodes 18A, 18B include a first electrode group 26A including a plurality of the first electrodes 18A and a second electrode group 26B including a plurality of the second electrodes 18B. Each first electrode 18A of the first electrode group 26A and each second electrode 18B of the second electrode group 26B have different polarities when electricity is output to the treated portion of the living body. For example, when the first electrodes 18A are positive electrodes, the second electrodes 18B are negative electrodes, and when the first electrodes 18A are negative electrodes, the second electrodes 18B are positive electrodes. When defibrillation is performed, for example, the first electrode group 26A is disposed in the coronary sinus and the second electrode group 26B is disposed in the right atrium. The plurality of electrodes 18A, 18B are provided spaced apart in the axial direction of the catheter shaft 14. Each first electrode 18A of the first electrode group 26A and each second electrode 18B of the second electrode group 26B of the present embodiment are disposed together. Note that the number of first electrodes 18A and the number of second electrodes 18B may be singular.

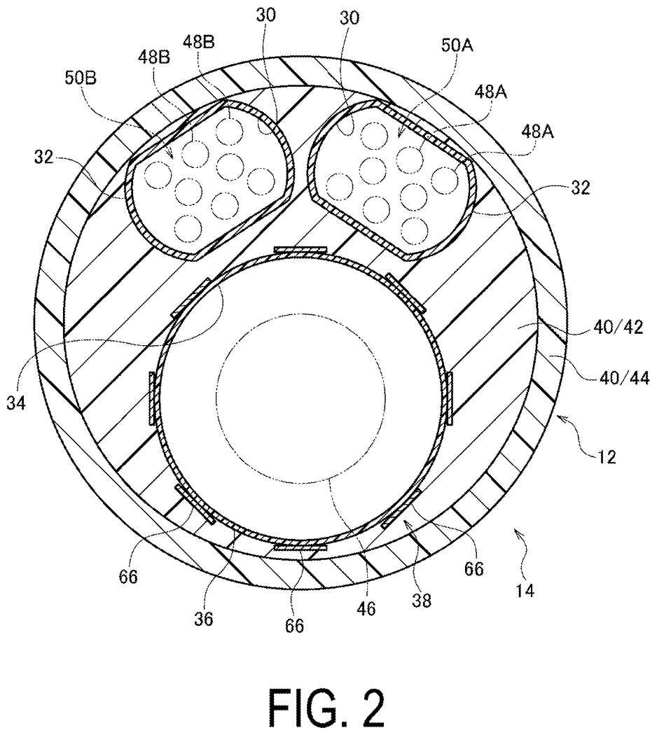

Referring to FIG. 2, the catheter shaft 14 includes, in addition to the shaft main body 12, a first lumen tube 32 that forms a first lumen 30 therein, a second lumen tube 36 that forms the second lumen 34 therein, and a reinforcement layer 38 embedded into the shaft main body 12. The reinforcement layer 38 will be described below.

The shaft main body 12 is inserted into an interior of a living body at least at the distal end side portion 12b, and is then disposed in a treated portion of the living body. The shaft main body 12 encloses the first lumen 30 and the second lumen 34 separated from each other. Here, “separated from each other” means that a double structure is not formed in which one of the first lumen 30 and the second lumen 34 is formed inside the other. The shaft main body 12 of the present embodiment also encloses the first lumen tube 32 and the second lumen tube 36. The shaft main body 12 of the present embodiment encloses two first lumens 30 and one second lumen 34, and each of the first lumens 30 is formed by an individual first lumen tube 32.

The shaft main body 12 is formed of at least one resin layer 40. The shaft main body 12 includes, as the resin layer 40 constituting the shaft main body 12, an inner layer 42 covering the first lumen tubes 32 and the second lumen tube 36, and an outer layer 44 covering the inner layer 42.

Each of the lumen tubes 32, 36 and the resin layer 40 (inner layer 42 and outer layer 44) of the shaft main body 12 are made of a synthetic resin having insulating properties. The lumen tubes 32, 36 are made of a synthetic resin having favorable slidability, such as polytetrafluoroethylene (PTFE) or perfluoro alkoxy alkane (PFA), for example. The outer layer 44 is made of a synthetic resin having favorable slidability, mechanical strength, flexibility, and biocompatibility, such as polyether block ether (PEBAX). The inner layer 42 is made of a synthetic resin, such as polyolefin, polyamide, polyether block amide (PEBAX or the like), polyurethane, or nylon. A contrast agent, a pigment, or the like may be mixed and kneaded into the inner layer 42 and the outer layer 44. A dielectric strength of the inner layer 42 may be, for example, lower than a dielectric strength of the outer layer 44.

The second lumen tube 36, on the proximal end side, extends outwardly of the shaft main body 12 in the axial direction, is inserted through an interior of the handle 16 and the second protective tube 20B, and is attached to the port member 24 (refer to FIG. 1). When the distal end side portion 12b of the shaft main body 12 is inserted into a living body, the second lumen 34 communicates an external space outside the living body and an internal space inside the living body. At this time, the second lumen 34 communicates with the external space of the living body through the port member 24 and communicates with the internal space of the living body through the distal end tip 14a. The second lumen 34 is regarded as open to the outside of the electrode catheter 10 on both end sides thereof. Accordingly, the second lumen 34 can be used as an insertion path of a cord-like member 46, such as a guide wire, or as a flow path of a fluid. When a guide wire is used as the cord-like member 46, an outer diameter of the guide wire is preferably within a range from 0.018 inches (0.46 mm) to 0.038 inches (0.97 mm), and more preferably within a range from 0.025 inches (0.63 mm) to 0.035 inches (0.89 mm).

When the second lumen 34 is used as an insertion path of the cord-like member 46, the cord-like member 46 inserted from the external space to the internal space of the living body is inserted through the second lumen 34. At this time, the cord-like member 46 (guide wire) may be indwelled in the internal space of the living body in advance, and the shaft main body 12 may be guided by the cord-like member 46 inserted through the second lumen 34 when the shaft main body 12 is inserted into the internal space of the living body. lternatively, the distal end side portion 12b of the shaft main body 12 may be indwelled in the internal space of the living body in advance, and when the cord-like member 46 is inserted into the second lumen 34 from the external space of the living body, the cord-like member 46 may be guided by the second lumen 34. When the second lumen 34 is used as a flow path of a fluid, the fluid flows from one of the external space and the internal space of the living body to the other through the second lumen 34. Here, the fluid refers to, for example, a liquid agent such as a contrast agent administered from the external space to the internal space of the living body.

In a cross section orthogonal to the axial direction of the catheter shaft 14, a cross-sectional area S2 of the second lumen 34 is larger than a cross-sectional area S1 of the first lumen 30. The cross-sectional area S2 of the second lumen 34 is the largest of all cross-sectional areas of the lumens inside the catheter shaft 14. Accordingly, when the second lumen 34 is used as an insertion path of the cord-like member 46, it is possible to increase the size of the cord-like member 46. In addition, when the second lumen 34 is used as a flow path of a fluid, the fluid flow rate can be increased. These are advantageous effects compared to a case in which the cross-sectional area S2 of the second lumen 34 is smaller than the cross-sectional area S1 of the first lumen 30.

The electrode catheter 10 includes a plurality of lead wires 48A, 48B respectively electrically conductive to the plurality of electrodes 18A, 18B. The plurality of lead wires 48A, 48B serve as conduction paths of the electricity supplied for the treatment and provided from the external power supply device to the plurality of electrodes 18A, 18B. The plurality of lead wires 48A, 48B include a first lead wire group 50A composed of the plurality of the first lead wires 48A and a second lead wire group 50B composed of the plurality of the second lead wires 48B. The first lead wires 48A are in one-to-one correspondence with the first electrodes 18A, and are electrically conductive to the corresponding first electrodes 18A. The second lead wires 48B are in one to-one correspondence with the second electrodes 18B, and are electrically conductive to the corresponding second electrodes 18B. The first lead wire group 50A and the second lead wire group 50B are inserted into the first lumens 30 that are different from each other. The plurality of lead wires 48A, 48B are inserted through the interior of the handle 16 and the first protective tube 20A in addition to the first lumens 30, and are respectively electrically conductive to a plurality of output terminals built into the connector 22.

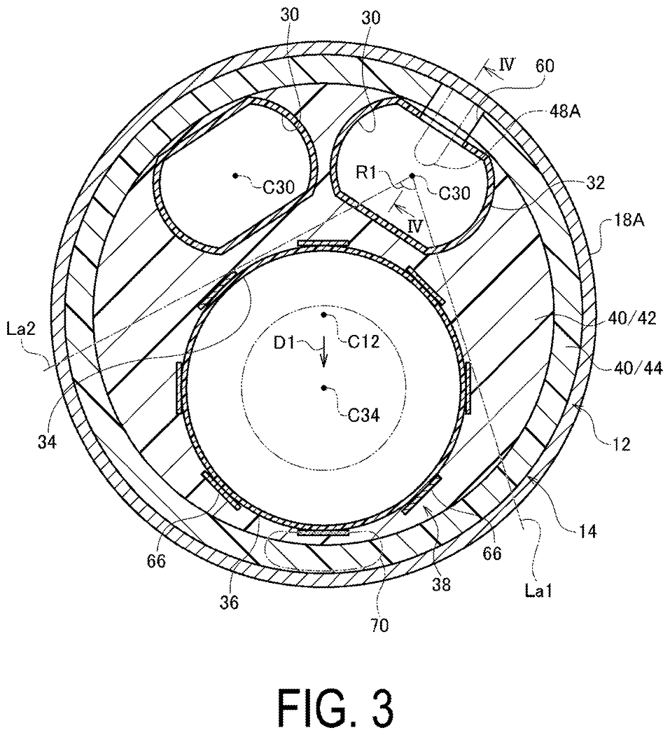

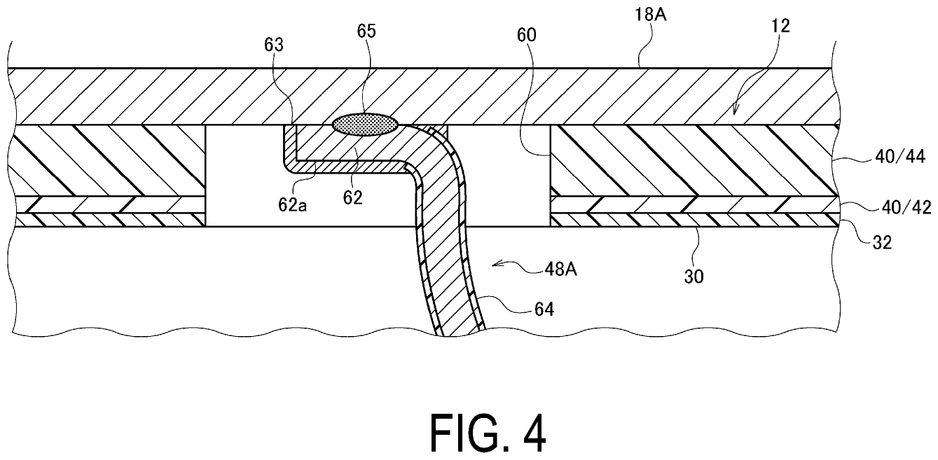

With reference to FIGS. 3 and 4, hereinafter, features common to the electrodes 18A, 18B and the lead wires 48A, 48B will be described using the first electrode 18A and the first lead wire 48A as examples. The electrodes 18A, 18B are mounted onto the outer peripheral portion of the shaft main body 12 in a state of close contact by being plastically deformed inwardly in a radial direction by a swaging process, for example. Side holes 60 individually corresponding to respective mounting positions of the plurality of electrodes 18A, 18B are formed in the catheter shaft 14. The side hole 60 is formed extending from an outer peripheral surface of the shaft main body 12 to the first lumen 30 at the mounting positions of the corresponding electrodes 18A, 18B. The lead wires 48A, 48B are drawn outside the first lumen 30 through the side hole 60 of the catheter shaft 14 and joined to inner peripheral surfaces of the electrodes 18A, 18B corresponding thereto, and are thus electrically conductive to the electrodes 18A, 18B.

Each of the lead wires 48A, 48B of the present embodiment includes a metal core wire 62 and an insulating layer 64 covering the metal core wire 62. The metal core wire 62 includes an exposed portion 62a not covered by the insulating layer 64 and exposed at distal end portions of the lead wires 48A, 48B. The lead wires 48A, 48B of the present embodiment are joined to the inner peripheral surfaces of the electrodes 18A, 18B by bonding, welding, or the like at the exposed portions 62a of the metal core wires 62. In the present embodiment, the lead wires 48A, 48B are bonded to the electrodes 18A, 18B by both bonding and welding. FIG. 4 illustrates an example in which an adhesive 63 for adhesion is applied to the lead wires 48A, 48B and the electrodes 18A, 18B, and penetration portions 65 are formed in the metal core wires 62 of the lead wires 48A, 48B and in the electrodes 18A, 18B by welding. Note that the insulating layer 64 on the lead wires 48A, 48B is not essential.

The reinforcement layer 38 is constituted by a braid obtained by braiding a plurality of element wires 66 into a tubular shape. In addition, the reinforcement layer 38 may be a coil or the like constituted by a single element wire 66. The reinforcement layer 38 has the role of enhancing a torque transmissibility of the catheter shaft 14. To realize this, the reinforcement layer 38 is embedded into the resin layer 40 constituting the shaft main body 12, and can transmit the torque applied from the handle 16 to the shaft main body 12 integrally with the shaft main body 12. The reinforcement layer 38 of the present embodiment is wound around the second lumen tube 36 and embedded into the resin layer 40 (inner layer 42) of the shaft main body 12 that covers the second lumen tube 36.

The reinforcement layer 38 is made of a material having conductivity. The material of the reinforcement layer 38 is not particularly limited and, in addition to a metal such as steel (stainless steel or the like) or aluminum, a resin having conductivity may be employed. The metal here also includes alloys including, as main components, the metals mentioned. By using a metal as the material of the reinforcement layer 38 as described above, it is possible to improve the torque transmissibility as compared with a case in which a resin is used as the material.

The reinforcement layer 38, inside the electrodes 18A, 18B having ring shapes, surrounds the second lumen 34 without surrounding the first lumens 30. The first lumens 30 are disposed on an outer side of the location surrounded by the reinforcement layer 38. Accordingly, the side hole 60 of the catheter shaft 14 can be provided about a center C30 of the first lumen 30 while avoiding a circumferential-direction range R1 including the entire reinforcement layer 38. Assume that a tangent line passing through the center C30 of the first lumen 30 and tangent to the reinforcement layer 38 from a first most circumferential side about the center C30 is a first tangent line La1, and a tangent line tangent to the reinforcement layer 38 from a second most circumferential side about the center C30 is a second tangent line La2. In this case, the circumferential-direction range R1 refers to a range from the first tangential line La1 to the second tangential line La2. Accordingly, when the lead wires 48A, 48B in the first lumens 30 are electrically conductive to the electrodes 18A, 18B, the lead wires 48A, 48B can be drawn from the first lumens 30 while avoiding locations surrounded by the reinforcement layer 38. In the present embodiment, the side hole 60 is provided on a side opposite to the second lumen 34 across the center C30 of the first lumen 30. The positional relationship described here is satisfied in a cross section orthogonal to the axial direction of the catheter shaft 14.

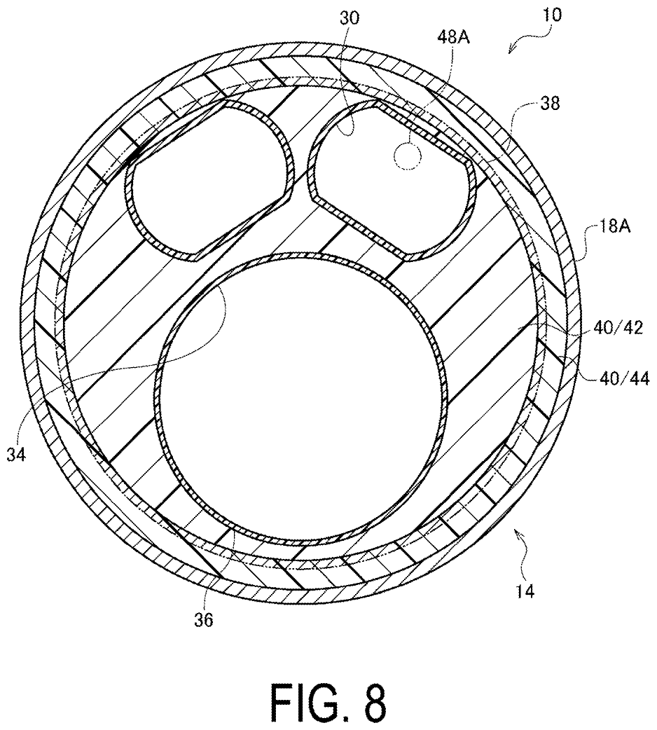

Effects of the electrode catheter 10 described above will now be described. FIG. 8 illustrates a portion of the electrode catheter 10 according to a reference embodiment. The electrode catheter 10 of the reference embodiment differs from the electrode catheter 10 of the embodiment in the position of the reinforcement layer 38 described in the following. Here, for convenience of description, a range in which the reinforcement layer 38 is embedded is indicated by a two dot chain line.

Consider a case in which the reinforcement layer 38 surrounds the plurality of lumens 30, 34. In this case, when the lead wires 48A, 48B in the first lumens 30 are electrically conductive to the electrodes 18A, 18B, the lead wires 48A, 48B need to be drawn from the first lumens 30 and thus pass through the location surrounded by the reinforcement layer 38. At this time, in a case in which the reinforcement layer 38 is constituted by a braid, a coil, or the like, the lead wires 48A, 48B are drawn from the first lumens 30 and thus pass through gaps between the element wires of the reinforcement layer 38. When the lead wires 48A, 48B come into contact with the reinforcement layer 38 due to positional deviation of the lead wires 48A, 48B in the first lumens 30 or the like, there is a concern that the reinforcement layer 38 and the lead wires 48A, 48B may unintentionally electrically conductive. This unintended conduction may occur, for example, when the metal core wires 62 (refer to FIG. 4) of the lead wires 48A, 48B are in direct contact with the reinforcement layer 38. The metal core wires 62 of the lead wires 48A, 48B are typically insulated by being covered with the insulating layer 64. Even if the lead wires 48A, 48B are insulated in this manner, in a case in which high-voltage electricity is output from the plurality of electrodes 18A, 18B, when the insulating layer 64 of the lead wires 48A, 48B and the reinforcement layer 38 are in contact with each other, the proximity of the metal core wires 62 of the lead wires 48A, 48B and the reinforcement layer 38 may cause insulation breakdown of the insulating layer 64. In a case in which such insulation breakdown occurs as well, there is a possibility of unintended conduction between the reinforcement layer 38 and the lead wires 48A, 48B.

Referring to FIG. 3, the reinforcement layer 38 of the present embodiment surrounds the second lumen 34 without surrounding the first lumens 30 through which the lead wires 48A, 48B are inserted. Accordingly, when the lead wires 48A, 48B in the first lumens 30 are electrically conductive to the electrodes 18A, 18B, the lead wires 48A, 48B can be drawn from the first lumens 30 while avoiding the location surrounded by the reinforcement layer 38. As a result, unintended conduction between the reinforcement layer 38 and the lead wires 48A, 48B due to contact between the reinforcement layer 38 and the lead wires 48A, 48B can be prevented. In addition, with the reinforcement layer 38 provided in the catheter shaft 14, the torque transmissibility can be ensured.

Further, the electrodes 18A, 18B of the present embodiment are used to output electricity supplied for treatment of the living body. In this case, as compared with a case in which the plurality of electrodes 18A, 18B are used to measure cardiac potential or the like, a potential difference generated between the plurality of electrodes 18A, 18B becomes extremely large, increasing the risk of insulation breakdown of the insulating layer 64. Even under such circumstances, there is an advantage that unintended conduction between the reinforcement layer 38 and the lead wires 48A, 48B can be prevented by the configuration described above.

Note that, when the electrode catheter 10 is used with the inferior vena cava approach, the electrode group 26A on the distal end side is indwelled in the coronary sinus and the electrode group 26B on the proximal end side is indwelled in the right atrium, forming a loop. By ensuring the torque transmissibility of the catheter shaft 14, it is possible to improve workability when forming a loop in the right atrium.

Other features of the electrode catheter 10 will now be described. Referring to FIG. 1, the reinforcement layer 38 is provided at least in an axial-direction range Ra1 continuous from the handle 16 to at least the electrodes 18A, 18B on the most proximal end side. The reinforcement layer 38 is preferably provided in an axial-direction range Ra2 continuous from the handle 16 to the electrodes 18A, 18B on the most distal end side. The reinforcement layer 38 of the present embodiment is provided in an axial-direction range continuous from the handle 16 to the distal end tip 14a. To satisfy this condition, the reinforcement layer 38 need not be provided up to the interior of the distal end tip 14a. Further, to satisfy this condition, in the interior of the handle 16, the reinforcement layer 38 need only be provided at a location where the torque is transmitted from the handle 16 to the catheter shaft 14. This makes it possible to effectively transmit the torque applied from the handle 16 to the shaft main body 12 by the reinforcement layer 38 to locations where the electrodes 18A, 18B are present.

Referring to FIG. 3, a center C34 of the second lumen 34 is eccentric to a center C12 of the shaft main body 12 in an eccentric direction D1. Here, the “center of the lumen” refers to a geometric center of a shape formed by an inner peripheral surface of the referred lumen. Further, the center C12 of the shaft main body 12 refers to a geometric center of a shape formed by the outer peripheral surface of the shaft main body 12. Thus, a narrow portion 70 extending from the electrodes 18A, 18B to the second lumen 34 is provided between a surface portion of the inner peripheral surface of the second lumen 34 in the eccentric direction D1 and the outer peripheral surface of the shaft main body 12. The centers C30 of the two first lumens 30 are eccentric to the center C12 of the shaft main body 12 in a direction opposite to the eccentric direction D1 of the second lumen 34, and are spaced apart in the circumferential direction of the catheter shaft 14.

The catheter shaft 14 is constituted by an integrally molded product. The molding method for obtaining this integrally molded product is not particularly limited. Here, a case in which extrusion-molding is used as the molding method will be described as an example. The resin layer 40 (here, inner layer 42) of the shaft main body 12 covering the lumen tubes 32, 36 is formed by this extrusion-molding. Thus, the shaft main body 12 can be provided integrally with the reinforcement layer 38 and the lumen tubes 32, 36. In the present embodiment, in addition to the lumen tubes 32, 36 of the catheter shaft 14, the outer layer 44 covering the inner layer 42 is also formed by extrusion-molding. A method of manufacturing this catheter shaft 14 will now be described.

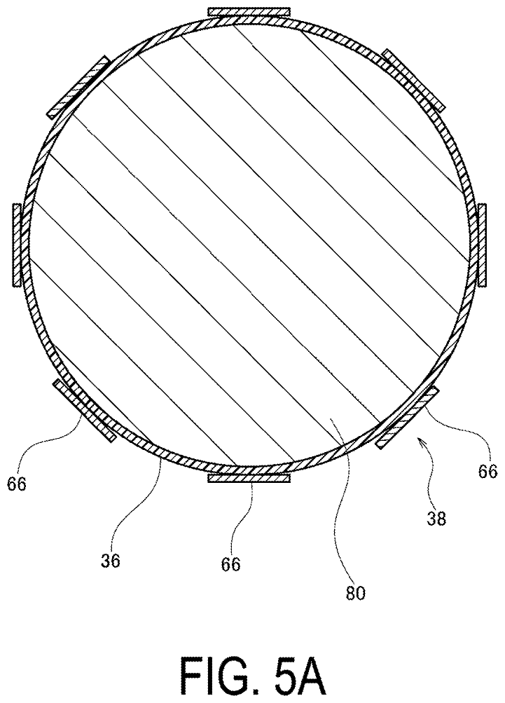

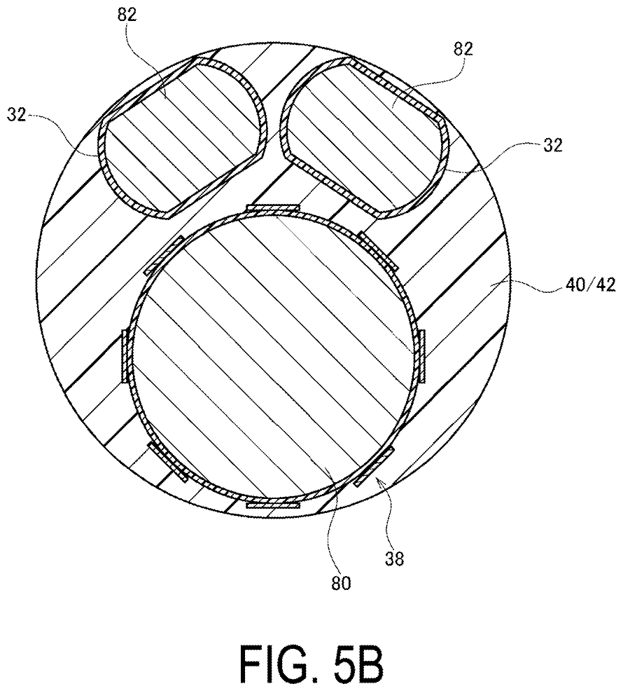

Referring to FIG. 5A, first, the second lumen tube 36 is disposed on an outer peripheral side of a mandrel 80 corresponding to the second lumen tube 36. At this time, the mandrel 80 may be covered with the second lumen tube 36 prepared in advance, or the second lumen tube 36 may be formed by extrusion-molding. Next, the reinforcement layer 38 is wound around an outer peripheral portion of the second lumen tube 36. At this time, for example, the reinforcement layer 38 is formed as a braid by braiding the plurality of element wires 66 into a tubular shape while feeding the element wires 66 from a braider.

Referring to FIG. 5B, next, with the reinforcement layer 38 wound around the second lumen tube 36, the inner layer 42 that covers the first lumen tubes 32 and the second lumen tube 36 is extrusion-molded, embedding the reinforcement layer 38. At this time, the inner layer 42 is extrusion-molded by extruding a molten resin constituting the inner layer 42 from an extruder. Prior to this, the first lumen tubes 32 are disposed on the outer peripheral side of mandrels 82 corresponding to the first lumen tubes 32. At this time, the mandrels 82 may be covered with the first lumen tubes 32 prepared in advance, or the first lumen tubes 32 may be formed by extrusion-molding.

Next, the outer layer 44 is extrusion-molded so as to cover the inner layer 42, thereby obtaining the catheter shaft 14 as a finished product. At this time, the outer layer 44 is extrusion-molded by extruding a molten resin, which is a material of the outer layer 44, from an extruder.

Here, the shaft main body 12 is composed of at least one resin layer 40 and encloses the second lumen tube 36 as well. In this case, as illustrated in FIG. 5B, when the resin layer 40 (here, inner layer 42) covering the second lumen tube 36 is extrusion-molded, a compressive load in the axial direction is applied to the second lumen tube 36 by the molten resin constituting the resin layer 40. This can result in a sagging deformation in which the second lumen tube 36 bulges radially outward away from the mandrel 80. As a countermeasure, typically, the second lumen tube 36 is maintained at a thickness which can sufficiently resist sagging deformation.

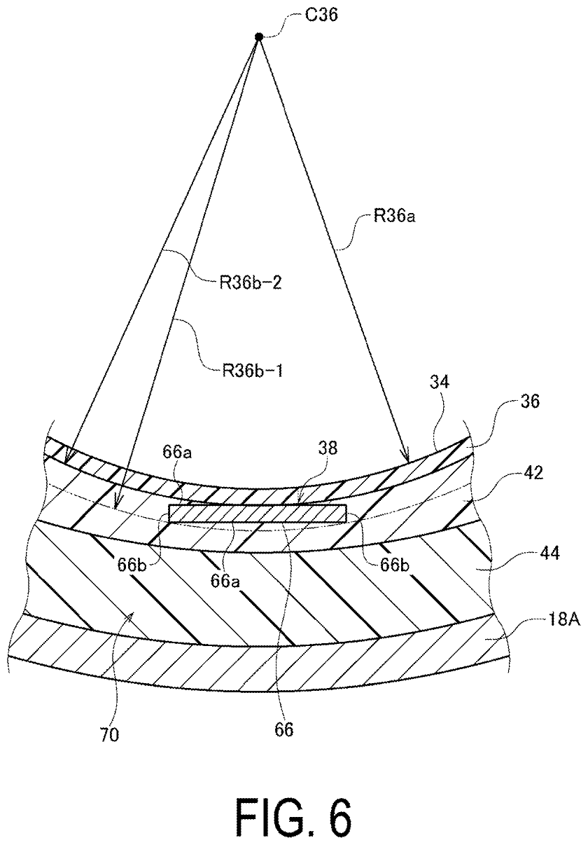

The reinforcement layer 38 of the present embodiment is wound around the second lumen tube 36 and embedded into the resin layer 40 covering the second lumen tube 36. Therefore, the sagging deformation of the second lumen tube 36 occurring at the time of extrusion-molding of the resin layer 40 covering the second lumen tube 36 can be restrained by the reinforcement layer 38, and the thickness required for the second lumen tube 36 to resist the sagging deformation can be reduced. Accordingly, compared to a case in which the reinforcement layer 38 wound around the second lumen tube 36 is not provided, an outer diameter of the second lumen tube 36 can be reduced while an inner diameter of the second lumen tube 36 remains unchanged. This means that, in terms of the example in FIG. 6, the outer diameter of the second lumen tube 36 is reduced from R36b-1 to R36b-2 while the inner diameter R36a of the second lumen tube 36 remains unchanged. Accordingly, compared to a case in which the reinforcement layer 38 wound around the second lumen tube 36 is not provided, a distance from the reinforcement layer 38 wound around the second lumen tube 36 to the electrodes 18A, 18B (particularly, a distance at the narrow portion 70 of the shaft main body 12) can be increased. Consequently, unintended conduction between the electrodes 18A, 18B and the reinforcement layer 38 due to insulation breakdown of the resin layer 40 of the shaft main body 12 (particularly, the outer layer 44 of the narrow portion 70) can be prevented. Note that the thickness of the second lumen tube 36 is, for example, within a range from 10 μm to 50 μm.

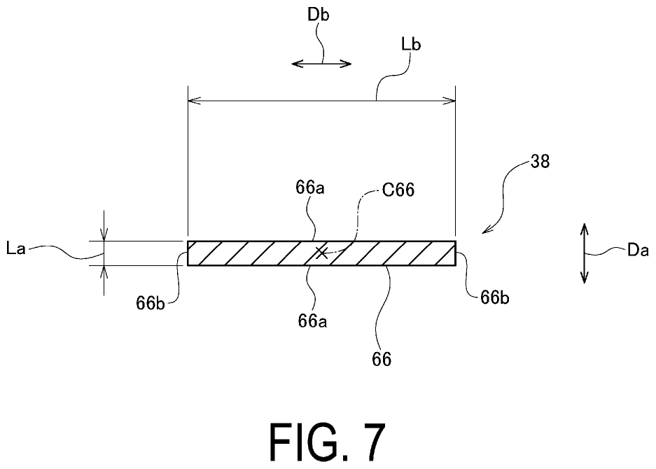

Referring to FIG. 7, each of the element wires 66 in the present embodiment is spirally wound about the second lumen tube 36, although not illustrated. In a cross section orthogonal to a center line passing through a center C66 of the element wire 66 constituting the reinforcement layer 38, a cross-sectional shape of the element wire 66 is a flat shape. This condition is satisfied in each of the element wires 66 of the braid. To realize this, a flat wire is adopted as the element wire 66 of the present embodiment, but a wire material having an oval shape or the like may be adopted. In the cross section orthogonal to the center line of the element wire 66, the smallest outer dimension of a straight line passing through the center C66 of the element wire 66 is referred to as a minor axial direction dimension La, and a direction along the straight line at that time is referred to as a minor axial direction Da. Further, a direction orthogonal to the minor axial direction Da of the flat shape formed by the element wire 66 is referred to as a major axial direction Db. A major axial direction dimension Lb along the major axial direction Db of the element wire 66 is, for example, preferably from 2 times to 10 times, more preferably from 3 times to 7 times, the minor axial direction dimension La. The minor axial direction dimension La of the element wire 66 is preferably 50 μm or less, more preferably 35 μm or less.

The element wire 66 includes a pair of long-side surface portions 66a positioned on both sides in the minor axial direction Da and extending in the major axial direction Db, and a pair of short-side surface portions 66b connecting the pair of long-side surface portions 66a. The long-side surface portions 66a of the present embodiment each have a linear shape extending in the major axial direction Db, but may have a curved shape. The short-side surface portions 66b of the present embodiment each have a linear shape connected to the long-side surface portions 66a via corner portions, but may have a curved shape smoothly connecting the pair of long-side surface portions 66a. In the present embodiment, one long-side surface portion 66a of at least one element wire 66 among the plurality of element wires 66 is in contact with the second lumen tube 36 in a cross section orthogonal to the axial direction of the catheter shaft 14 (refer to FIG. 6).

When the catheter shaft 14 is manufactured, the plurality of element wires 66 serving as the reinforcement layer 38 are wound about the second lumen tube 36, and the resin layer 40 (here, inner layer 42) of the shaft main body 12 is extrusion-molded, embedding the reinforcement layer 38. At this time, by using the element wire 66 having such a flat shape, the long-side surface portions 66a of the element wire 66 can be disposed at positions facing the outer peripheral surface of the second lumen tube 36 in the radial direction around the second lumen 34. In other words, the outer peripheral surface of the second lumen tube 36 can be disposed at a position facing the minor axial direction Da of each element wire 66.

This makes it easier to reduce a radial-direction dimension of the element wire 66 about the second lumen 34 to the extent possible compared to a case in which the element wire 66 having a circular shape with the same cross-sectional area is employed, and the element wire 66 can be brought closer to the second lumen tube 36 to that same extent. Consequently, a distance from the reinforcement layer 38 to the electrodes 18A, 18B (particularly, the distance at the narrow portion 70 of the shaft main body 12) can be increased without changing an inner shape of the second lumen 34 and an outer shape of the shaft main body 12. Consequently, unintended conduction between the electrodes 18A, 18B and the reinforcement layer 38 due to insulation breakdown of the resin layer 40 of the shaft main body 12 (particularly, the resin layer 40 of the narrow portion 70) can be prevented.

Next, modifications of the constituent elements described above will be described.

The catheter shaft 14 need not include each lumen tube 32, 36, or may include only one of the first lumen tube 32 and the second lumen tube 36. The shaft main body 12 may enclose a lumen other than the first lumen 30 and the second lumen 34. The quantity of the second lumens 34 is not particularly limited, and may be one or three or more. The resin layer 40 of the shaft main body 12 may be only the outer layer 44, or may include three or more layers. The cross-sectional area S1 of the first lumen 30 may be the same as the cross-sectional area S2 of the second lumen 34, or may be smaller than the cross-sectional area S2. The second lumen 34 may be closed and not open toward the outside of the electrode catheter 10 on both end sides thereof. In this case, a member other than the lead wires 48A, 48B may be inserted into the second lumen 34, or a specific member may not be disposed.

The reinforcement layer 38 may be, in addition to the braid and the coil, a tube or the like having conductivity. The reinforcement layer 38 need only be provided within an axial-direction range including at least one of the electrodes 18A, 18B. For example, the reinforcement layer 38 need not be provided within the axial-direction range from the electrodes 18A, 18B on the most distal end side to the distal end tip 14a. The cross-sectional shape of the element wire 66 of the reinforcement layer 38 is not particularly limited, and may be a circular shape.

As a molding method of the catheter shaft 14, reflow molding may be used in which each resin layer 40 is molded by using a material tube that is a material of the resin layer 40 and a heat-shrinkable tube. In such reflow molding, the mandrel is covered with the lumen tubes 32, 36, the lumen tubes 32, 36 are covered with a material tube that is a material of the inner layer 42, and the material tube is further covered with a heat-shrinkable tube. At this time, the reinforcement layer 38 is wound around the second lumen tube 36, and then the lumen tubes 32, 36 are covered with the material tube. Subsequently, the heat-shrinkable tube and the material tube are heated, and the molten resin obtained by melting the material tube is brought into close contact with each of the lumen tubes 32, 36 by heat shrinkage of the heat-shrinkable tube. Subsequently, when the heat-shrinkable tube and the molten resin are cooled, the inner layer 42 can be molded by solidification of the molten resin. Simultaneously with or subsequent to this cooling, the heat-shrinkable tube can be peeled from the inner layer 42 by tearing or the like. Next, the inner layer 42 covering each of the lumen tubes 32, 36 is covered with a material tube that is a material of the outer layer 44, and the material tube is further covered with a heat-shrinkable tube. Subsequently, the outer layer 44 can be formed in the same manner as described above by heating the heat-shrinkable tube and the material tube, cooling the heat-shrinkable tube and the molten resin, and peeling off the heat-shrinkable tube, in this order, thereby forming the catheter shaft 14.

Note that, when reflow molding is used as the molding method of the catheter shaft 14, typically, the plurality of lumen tubes 32, 36 are in contact with each other, and no gap is provided therebetween. In contrast, when the extrusion-molding described above is used as the molding method of the catheter shaft 14, a gap is typically provided between the plurality of lumen tubes 32, 36. In the embodiment, a gap is provided between the adjacent first lumen tubes 32 and between the first lumen tubes 32 and the second lumen tube 36. A distance of this gap is preferably within a range from 10 μm to 300 μm, more preferably within a range from 10 μm to 200 μm, at a location where a distance between the adjacent lumen tubes 32, 36 is the narrowest.

The embodiments and modifications described above are examples. Technical ideas obtained by abstraction thereof should not be interpreted as limited to the contents of the embodiments and modifications. Numerous design changes, such as modification, addition, and deletion of constituent elements, can be made to the contents of the embodiments and modifications. In the embodiments described above, the content in which such design changes can be made has been emphasized with expressions such as “of the embodiment.” However, design changes are also possible even in the content without such an expression. Hatching in sections of the drawings does not limit the material of a hatched object. Structures and numerical values referred to in the embodiments and the modifications naturally include those that can be regarded as the same in consideration of manufacturing errors and the like.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An electrode catheter comprising:
a catheter shaft including
a shaft main body enclosing a first lumen and a second lumen separated from each other, and
a reinforcement layer embedded into the shaft main body;
an electrode having a ring shape and mounted to an outer peripheral portion of the shaft main body; and
a lead wire electrically conductive to the electrode and inserted into the first lumen, wherein
the reinforcement layer has conductivity and, inside the electrode, surrounds the second lumen without surrounding the first lumen.

2. The electrode catheter according to claim 1, wherein
the catheter shaft includes a lumen tube forming the second lumen,
the shaft main body is formed of at least one resin layer, and
the reinforcement layer is wound around the lumen tube and is embedded into the at least one resin layer covering the lumen tube.

3. The electrode catheter according to claim 2, wherein
the at least one resin layer covering the lumen tube is formed by extrusion-molding.

4. The electrode catheter according to claim 1, wherein
the reinforcement layer is constituted by a braid obtained by braiding a plurality of element wires into a tubular shape, and
a cross-sectional shape of each of the plurality of element wires is a flat shape in a cross section orthogonal to a center line of the element wire.

5. The electrode catheter according to claim 1, wherein
the catheter shaft is formed with a side hole extending from an outer peripheral surface of the shaft main body to the first lumen at a mounting position of the electrode, and
the side hole is provided about a center of the first lumen while avoiding a circumferential-direction range including the entire reinforcement layer.

6. The electrode catheter according to claim 1, comprising
a handle attached to a proximal end side portion of the shaft main body, wherein
the reinforcement layer is provided at least in an axial-direction range continuous from the handle to the electrode.

7. The electrode catheter according to claim 1, wherein
the second lumen communicates an external space outside a living body and an internal space inside the living body when a distal end side portion of the shaft main body is inserted into the living body.

8. The electrode catheter according to claim 1, wherein
the electrode is used to output electricity supplied for treatment of a treated portion of a living body.

9. The electrode catheter according to claim 1, wherein
a cross-sectional area of the second lumen is larger than a cross-sectional area of the first lumen in a cross section orthogonal to an axial direction of the catheter shaft.

10. The electrode catheter according to claim 1, wherein
a material of the reinforcement layer is a metal.

11. A method of manufacturing the catheter shaft according to claim 2, the method comprising:
extrusion-molding the at least one resin layer covering the lumen tube and thus embedding the reinforcement layer, in a state where the reinforcement layer is wound around an outer peripheral portion of the lumen tube.
